(19) United States
(12) Patent Application Publication
(10) Pub. No.: US 2004/0043047 A1
(43) Pub. Date: Mar. 4, 2004

(54) Cosmetic or dermatological compositions containing at least one substance for increasing the functionality and/or expression of the CD44 membrane receptors of skin cells

(75) Inventors: Marc Dumas, Orleans (FR); Frederic Bonte, Orleans (FR)

Correspondence Address: Gary M. Nath, NATH & ASSOCIATES PLLC, 6th Floor, 1030 15th Street, N.W., Washington, DC, 20005, US

(73) Assignee: Parfums Christian Dior, Paris (FR)

(21) Appl. No.: 10/650,965

(22) Filed: Aug. 29, 2003

Related U.S. Application Data
(63) Continuation of application No. 09/937,507, filed on Sep. 26, 2001, now abandoned.
Application No. 09/937,507 is a 371 of international application No. PCT/FR00/00764, filed on Mar. 27, 2000.

(30) Foreign Application Priority Data
Mar. 26, 1999 (FR) 99 03840

(51) Int. Cl. A61K 7/00

(52) U.S. Cl. 424/401

(57) ABSTRACT

The invention relates to uses in the cosmetic or pharmaceutical field of at least one active agent which increases the expression and/or the functionality of the. CD44 membrane receptors of skin cells and/or which improves the binding to the surface of the said skin cells of hyaluronic acid, and/or of collagen, in particular of collagen I and/or collagen IV, and/or fibronectin.

These active agents are preferably α-hydroxy acids or α-keto acids or salts or esters of these acids, or manganese chloride.

The cosmetic or pharmaceutical compositions of the invention are intended for improving the binding of hyaluronic acid and/or of collagen, in particular collagen I and/or collagen IV, and/or of fibronectin to the surface of skin cells, and especially for improving the moisturization of the dermis and the epidermis, for preventing or treating the phenomena of ageing of the skin and also inflammatory phenomena.

The invention relates to cosmetic or dermatological compositions containing at least one substance for increasing the functionality and/or expression of the CD44 membrane receptors of skin cells.

Hyaluronic acid is present in abundance in the human epidermis, where it is located in the intercellular spaces between the keratinocytes.

Hyaluronic acid is also present in abundance in the human dermis, where it is located in the intercellular spaces between the fibroblasts.

The glycoprotein known as CD44 is present in the epidermis at the surface of the keratinocytes and its function is to allow the binding of hyaluronic acid to the surface of the keratinocytes.

The glycoprotein known as CD44 is also present in the dermis at the surface of the fibroblasts and its function is to allow the binding of hyaluronic acid, and collagens, in particular collagens I and IV, and also fibronectin to the surface of these cells.

Hyaluronic acid is the natural water uptake agent of the epidermis and the dermis, since it is capable of retaining up to one thousand times its volume of water. It is thus clear to see from this the importance for the cell of having receptors capable of binding this water uptake agent in order to retain water on contact with the cell and to allow hydration of the cell.

It is moreover known that deposits of hyaluronic acid coincide with intense cell renewal and migration activity in various growing tissues and organs. In the epidermis, it is located in the living deep layers, at the basal and suprabasal level, where the cell renewal and migration are greatest. However, in the places where cellular adhesion to the matrix constituent is essential, for instance at the interface between the epidermal basal cells and the dermo-epidermal junction, CD44 is absent.

Another important element for the physiology of the epidermis and the dermis is that, in an inflammatory situation, the regions invaded by the inflammatory cells, lymphocytes and neutrophils, show a disappearance of CD44 and of hyaluronic acid. This disappearance is necessary for strong adhesion of the inflammatory cells and for their invasion and installation in the tissues (CD44 Substituted with Heparan Sulfate an Endo-β-galactosidase-Sensitive Oligosaccharides: A Major Proteoglycan in Adult Human Epidermis. J. Invest. Dermatol., 1997, 109, pp. 213-218). It is moreover known that hyaluronic acid makes it possible to

establish actual bridges between two CD44 receptors carried by adjacent cells. This is supported by the fact that, when a skin maintained under survival conditions is treated with hyaluronidase which degrades hyaluronic acid, the intercellular spaces are opened and acanthosis, that is to say an epidermal hyperplasia, is brought about. Such an acanthosis with disappearance of CD44 is also observed in the epidermal sites invaded by leukocytes.

These data together prove that CD44 has an important role in the development, maintenance and functionality of the epidermis.

The inventors have furthermore observed a decrease in binding between the CD44 receptors and hyaluronic acid with age. This phenomenon is the cause of the increase in dryness of the epidermis with age, and also of the decrease in epidermal cell renewal. Specifically, the epidermis becomes atrophied and renews less well with age.

From all this knowledge, it emerges that any means for increasing the functionality of the CD44 transmembrane receptors and thus for improving the binding of hyaluronic acid to the cell surface constitutes an advantageous means for improving the cellular hydration of the epidermis and the dermis.

Moreover, any means for intensifying the interconnection network between the epidermal cells by binding hyaluronic acid to CD44 receptors of two adjacent cells constitutes a particularly effective means for limiting the migration of inflammatory cells such as lymphocytes and neutrophils towards the epidermal cells. Such an action would have the effect of preventing or treating inflammation phenomena. Thus, the invention is of particular advantage in caring for sensitive and reactive skin.

Finally, it emerges that any means for increasing the expression and/or functionality of the CD44 transmembrane receptors and thus of improving the binding of hyaluronic acid to the cell surface constitutes an advantageous means for combating or, at least, slowing down and/or delaying the onset of the signs of ageing of the skin, in particular such as wrinkles, dryness of the skin, impairment of the complexion, impairment of the biomechanical properties of the skin such as slackening, and loss of tonicity, firmness and elasticity of the skin.

The present invention results from the demonstration, after a systematic study, that certain substances act effectively on functionality and expression of the CD44 receptors of epidermal cells. This discovery has led the inventors of the present invention to introduce the substances whose activity was thus demonstrated in cosmetic or dermatological compositions, intended in particular for maintaining or restoring the cellular hydration of the epidermis, for slowing down and/or delaying the onset of the signs of ageing of the skin, and for treating or preventing inflammation phenomena, in particular for caring for sensitive and reactive skin.

Thus, the invention results essentially from the discovery that it is possible to act, via cosmetic or pharmaceutical active agents for topical use, on the expression and/or functionality of the CD44 membrane receptors of skin cells, which has made it possible to envisage the use of these active agents in cosmetic or therapeutic treatment processes for topical use on the skin.

The expression “increasing the expression” means that an increase in the amount of CD44 receptors manufactured by the cells takes place.

For the purposes of the invention, the expression “increasing the functionality of the CD44 receptors” means that there is improvement in the binding, at the surface of the cells under consideration, of hyaluronic acid and/or of collagen, in particular of collagen I and/or collagen IV, and/or of fibronectin.

The invention thus relates to uses both in the cosmetic field and in the pharmaceutical field using this discovery. Needless to say, a person skilled in the art will select active agents that are acceptable in the intended field, depending on whether they are for cosmetic or pharmaceutical applications.

Thus, according to one of its essential aspects, the invention relates to the use, in a cosmetic composition, of at least one active agent intended to increase the expression and/or to improve the functionality of the CD44 membrane receptors of skin cells.

More specifically, according to this first aspect, the invention relates to the use, in a cosmetic composition, of at least one active agent which increases the expression of the CD44 membrane receptors of skin cells and/or which improves the binding to the surface of the said skin cells of hyaluronic acid, and/or of collagen, in particular of collagen I and/or collagen IV, and/or of fibronectin.

According to a first variant, the use of this active agent makes it possible to improve the functionality of the CD44 membrane receptors of skin cells in that they bind, to the surface of the said cells, hyaluronic acid and/or collagen, in particular collagen I and/or collagen IV and/or fibronectin.

It has been found that this action on the CD44 membrane receptors is experienced on the CD44 receptors of both keratinocytes and fibroblasts.

Thus, according to a first variant, the said active agent increases the expression of the CD44 receptors of keratinocytes and/or fibroblasts and/or improves the binding of hyaluronic acid to the surface of these cells.

According to another variant, the said active agent increases the expression of the CD44 receptors of fibroblasts and/or improves the binding, to the surface of the said fibroblasts, of collagen, in particular of collagen I and/or collagen IV, and/or of fibronectin.

As disclosed above, the invention results from a systematic study of a large number of active agents. This study has made it possible to show, in particular, that the active agents which are most effective on the expression and/or functionality of the CD44 membrane receptors of skin cells are found to be:

α-hydroxy acids, which will be chosen, needless to say, in the context of the invention, from those which are cosmetically acceptable, salts thereof and esters thereof. α-hydroxy acids which will be advantageously chosen are C₂ to C₁₂ α-hydroxy acids,

cosmetically acceptable α-keto acids, advantageously C₃ to C₁₂ α-keto acids, salts thereof and esters thereof.

Another product which is particularly effective is manganese chloride.

When an α-hydroxy acid or a salt or ester thereof is used, this acid is preferably chosen from the group consisting of lactic acid, glycolic acid, gentisic acid, salicylic acid, gluconic acid and heptonic acid.

When an α-keto acid is used, pyruvic acid is preferably chosen.

The α-hydroxy acid esters or α-keto acid esters are advantageously chosen from the esters thereof with a C₂-C₂₄ and preferably a C₁₄-C₂₂ alcohol.

The α-hydroxy acid salts or α-keto acid salts are advantageously chosen from the sodium, calcium, magnesium, zinc and manganese salts.

Finally, as emerges from the examples, it is seen that calcium gluconate is especially advantageous, as is gluconic acid combined with a calcium salt, for example with calcium chloride.

It also emerges from the examples that, when gluconate is used in the presence of calcium, the molar ratio of calcium to gluconate is advantageously between 2 and 6 and preferably about 4.

The proportions of active agents in the cosmetic compositions of the invention may vary within a wide range. However, it is advantageous to use between 0.005% and 5% by weight and preferably between 0.05% and 0.5% of active agent relative to the total weight of the final cosmetic composition.

Advantageously, the said active agent is used in the composition in combination with at least one substance chosen from the group consisting of vitamins, in particular vitamins of group A (retinol), C and D3 and derivatives thereof such as esters, in particular the palmitates and propionates, tocopherols, xanthines, in particular caffeine or theophylline, retinoids, in particular vitamin A acid, extracts of Centella asiatica, asiatic acid and madecassic acid and glycosyl derivatives thereof, such as asiaticoside or madecassoside, extracts of Siegesbeckia orientalis, extracts of Commiphora mukul and extracts of Eriobotrya japonica, cosmetically acceptable silicon derivatives such as polysiloxanes, silanols and silicones, amino acids and salts thereof, especially the magnesium or calcium salts, in particular aspartic acid, arginine, citrulline and threonine, ceramides, glycoceramides, sphingosine derivatives, in particular ceramides of type II and type III, phospholipids, forskolin and derivatives thereof, extracts of Coleus, extracts of Tephrosia, elastase inhibitors, in particular ellagic acid, soybean peptides, collagenase inhibitors, in particular peptides and plant extracts such as extracts of Coptidis root, extracts of Scutellaria baicalensis Georgi root, flavonoids such as wogonine, baicaline and baicaleine, aqueous-ethanolic extracts of leaves of Ginkgo biloba, of Mosla chinensis, of Salvia officinalis, of Cinnamomum cassia, cathechic extracts of Camellia sinensis and aqueous extracts of Theobroma cacao bean husks, extracts of Phellodendron amurense, anti-inflammatory agents, in particular phospholipase A2 inhibitors, calmants, in particular extracts of liquorice, glycyrrhetinic acid, ammonium glycyrrhizinate, moisturizers, in particular polyols, propylene glycol, butylene glycol, glycerol, hyaluronic acid, anti-stretch mark agents, in particular extracts of common horse chestnut and escin, agents for protecting or improving the microcirculation, in particular bioflavonoids from Ginkgo biloba, isodon, extracts of Ami visnaga, visnadine, ruscogenin, free-radical scavengers, in particular polyphenols such as PCOs (Procyanidol Oligomers) and derivatives thereof, plant extracts, in particular extracts of Curcuma longa, anti-seborrhoeic agents such as a 5-α-reductase inhibitor, in particular an extract of Pygeum africanum, and agents for stimulating the blood microcirculation, such as cepharanthine and methyl nicotinate, alfalfa saponins, soya sapogenols, in particular of type A and type B, agents for stimulating collagen VII synthesis, such as extracts of Bertholletia, ellagic acid, D-xylose and trace element complexes.

Advantageously, the said active agent is used in the composition in combination with at least one substance chosen from substances for protecting the skin against the harmful effects of sunlight, such as sunscreens alone or in combination, in particular UVA screening agents and UVB screening agents, in particular titanium oxides and zinc oxides, oxybenzone, cinnamates, in particular octyl methoxycinnamate (sold under the brand name Parsol MCX), butylmethoxydibenzoylmethane (sold under the brand name Parsol 1789) and screening agents of plant origin, substances for limiting the damage caused to DNA, in particular those limiting the formation of thymine dimers, such as ascorbic acid and derivitaves thereof and/or Photonyl, and substances for contributing towards removing age spots, such as inhibitors of melanin synthesis or tyrosinase inhibitors.

According to another of its essential characteristics, the invention relates to a cosmetic care method. This method is intended more particularly for correcting a deficiency in the expression and/or functionality of the CD44 membrane receptors of skin cells.

More specifically, the cosmetic care method according to the invention comprises the application to the areas of skin to be treated of a cosmetic composition containing, as active principle, at least one agent for increasing the expression of the CD44 membrane receptors of skin cells and/or for improving the binding to the surface of the said skin cells of hyaluronic acid, and/or of collagen, in particular of collagen I and/or collagen IV, and/or of fibronectin.

According to the method of the invention, the active principle will advantageously be chosen from α-hydroxy acids, α-keto acids and the salts thereof and derivatives thereof mentioned above.

Manganese chloride may also be chosen as an active principle of the cosmetic composition used in the cosmetic care process of the present invention.

In this cosmetic process, a cosmetic composition containing one of the active agents as defined above is applied to the part of the skin to be treated.

This cosmetic care method makes it possible especially to correct the deficiency in the binding to the surface of the skin cells of hyaluronic acid and/or of collagen, in particular of collagen I and/or collagen IV, and/or of fibronectin.

This cosmetic care method makes it possible in particular to improve the hydration of the epidermis, or to treat sensitive or reactive skin.

The invention also relates to a cosmetic care method as defined above, for correcting the negative effects of ageing on the expression and/or functionality of the CD44 membrane receptors of skin cells. This point is particularly important since it has been demonstrated by the inventors of the present invention that the ability of keratinocytes to bind hyaluronic acid decreases considerably in the course of ageing, thus reflecting a loss of functionality of CD44.

According to another of its aspects, the invention also relates to the uses in the therapeutic field of the discovery made by the inventors of the present invention.

Thus, the invention is also directed towards a therapeutic treatment process by applying to the skin a pharmaceutical composition for topical use containing, as active principle, at least one active agent for increasing the expression of the CD44 membrane receptors of skin cells and/or for improving the binding of hyaluronic acid and/or of collagen, in particular of collagen I and/or collagen IV, and/or of fibronectin to the surface of these cells.

The invention is directed more particularly towards the case in which the pharmaceutical compositions contain, as active agent, an active agent chosen from pharmaceutically acceptable α-hydroxy acids, salts thereof and esters thereof with a C₂-C₂₄ and preferably C₁₄-C₂₂ alcohol, pharmaceutically acceptable α-keto acids, salts thereof and esters thereof with a C₂-C₂₄ and preferably C₁₄-C₂₂ alcohol, and manganese chloride.

In these various therapeutic applications, the active agents will be chosen, needless to say, from those which are pharmaceutically acceptable. However, all the comments made in the disclosure hereinabove regarding α-hydroxy acids or α-keto acids, and the preferred salts and esters thereof, remain valid.

The concentrations of the active products in the compositions are also chosen within the same preferential ranges as those of the cosmetic active products in the cosmetic compositions described above.

Thus, according to another aspect, the invention relates to the use of at least one active agent chosen from pharmaceutically acceptable α-hydroxy acids, salts thereof and esters thereof with a C₂-C₂₄ and preferably C₁₄-C₂₂ alcohol, pharmaceutically acceptable α-keto acids, salts thereof, esters thereof with a C₂-C₂₄ and preferably C₁₄-C₂₂ alcohol, and manganese chloride, for the preparation of a pharmaceutical composition for topical use, for treating a deficiency in the expression of the CD44 receptors of skin cells and/or their ability to bind hyaluronic acid and/or collagen, in particular collagen I and/or collagen IV, and/or fibronectin to the surface of the said cells.

One of the particularly advantageous fields is that of the prevention and/or treatment of inflammatory phenomena.

The examples which follow are given for the purposes purely of illustrating the present invention.

### EXAMPLE 1

Test Protocol for Demonstrating by Fluorescence the Binding of Hyaluronic Acid to the Surface of Keratinocytes

I-Measurement of the Amount of Haluronic Acid Bound to the Surface of Keratinocytes

The following steps are performed:

1—Inoculation of Cells:

15,000 human keratinocytes are inoculated in culture wells (96-well plates) in a K-SFM medium (Gibco).

2—Production of Confluent Cultures:

Incubation of the cultures for 72 hours at 37° C. under a humidity-saturated atmosphere, with 5% CO₂, with renewal of the culture medium after 48 h.

3—Binding of Hyaluronic Acid:

Incubation of the cells for 4 hours at 4° C. with hyaluronic acid covalently bonded to a fluorochrome, fluorescamine (HA-FA), synthesized in the laboratory according to the technique described in “Preparation and properties of fluoresceine-labelled hyaluronate; carbohydrate research, 1975, Vol. 44, pp. 251-257”, prepared in K-SFM containing 0.2% sodium azide.

4—Evaluation of the Activity of the Test Products:

The products for which it is desired to evaluate the action on the amount of HA-FA bound by keratinocytes are added to the incubation medium at the start of incubation. Controls are carried out in the absence of the products to be evaluated and in the presence of their excipient which was used to dissolve them.

5—Nonspecific Binding of HA-FA.

In order to determine the nonspecific labelling, the same experimental conditions as in step 3 are rigorously used, but with the addition of 200 times more hyaluronic acid not labelled with a fluorochrome.

6—Fluorescence Quantification:

After the HA-FA binding medium has been removed, two rinses with PBS are carried out, after which the fluorescence bound to the keratinocytes is measured at an excitation wavelength λex 485 nm and an emission wavelength λem=538 nm.

7—Expressing the Results:

The results are expressed in fluorescence units (“specific fluorescence”), with the nonspecific fluorescence being deducted (see 5-).

8—Normalization:

In certain cases, in particular when it is desired to compare two different strains of cells, for example originating from donors of different ages, or from donors who have received a different treatment, it is necessary to normalize, taking account of the number of cells present in the culture wells. To do this, the fluorescence values measured are reduced to the cellular DNA content, carried out according to the DAPI method (see III-).

II—Measurement of the Expression of the Hyaluronic Acid Receptor (CD44) at the Surface of Keratinocytes

Steps 1 and 2 are identical to steps 1 and 2 of I.

3—Binding of the cells to 2% paraformaldehyde in PBS containing calcium and magnesium for 5 minutes at ambient temperature.

4—Saturation of the Nonspecific Antigen-Antibody Binding Sites:

After rinsing twice with PBS, the cells are incubated with PBS saturation medium with calcium and magnesium supplemented with 5% v/v of calf serum and 0.05% w/v of sodium azide, for 1 hour at ambient temperature.

5—Labelling:

Addition of R-phycoerythrin-coupled human anti-CD44 IgG1 monoclonal antibody diluted in the saturation medium (see 4-) 50-fold, for 1 hour at ambient temperature and in the absence of light.

6—An isotypic control with an R-phycoerythrin-coupled IgG1 is prepared under the same experimental conditions as for the labelling. The values obtained are subtracted from the labelling values.

7—Fluorescence Quantification:

After rinsing twice with the saturation medium, the fluorescence present in the wells is read with 50 μl of PBS at λ excitation=544 nm and k emission=590 nm.

8—Evaluating the Test Products:

The products whose activity on the expression of the CD44 receptor it is desired to evaluate are added to the culture medium 24 hours after inoculation of the cells. To do this, the inoculation medium is removed and replaced with an identical medium containing the test substance. The incubation then continues for 48 hours. Controls are carried out in the absence of the products to be evaluated, and in the presence of the excipient which was used to dissolve them. All the factors are otherwise equal.

9—Measurement of the functionality of the CD44 receptor. After treating the cultures for 48 hours with the substances to be evaluated (see 8-), the capacity of the cells to bind hyaluronic acid is measured according to protocol I-, by adding in step 3 of this protocol, CaCl₂ at a concentration of 20 mM.

10—Normalization:

The values thus obtained are normalized, taking account of the amount of cellular material present in the wells. To do this, the cellular DNA content is measured by the DAPI method (see protocol below).

III—Quantification of the Cellular DNA with 4′,6′-diaminophenylindole Dihydrochloride (DAPI)

The quantification of the cellular DNA may be carried out directly on the cultures after quantification of the CD44 expression.

1—Permeabilization Step:

30 μl of methanol at −20° C. are added to each well for 1 min.

2—Labelling with DAPI:

After rinsing twice with PBS, a 20 μg/ml DAPI solution is added over 15 minutes at ambient temperature and in darkness.

3—Fluorescence Quantification:

After rinsing twice with PBS, the fluorescence present in the wells is read with 50 μl of PBS at λ excitation=355 nm and λemission=460 nm.

### EXAMPLE 2

Demonstration of the Effect of Ageing on the Functionality of the CD44 Receptor in its Binding of Hyaluronic Acid

This study was performed using the test whose protocol (I-) is given in Example 1.

The study was performed on cultures of normal human keratinocytes obtained from donors of different ages.

The results are given in Table I below.

TABLE I

| | Binding of hyaluronic acid (λem = 538 nm) | |
|---|---|---|
| Ages of donors, | relative to the amount of DNA (λem = 460 nm) | |
| in years | Mean | Standard deviation |
| 18 | 257 | 13 |
| 20 | 505 | 42 |
| 33 | 378 | 52 |
| 44 | 75 | 21 |
| 50 | 77 | 2 |
| 67 | 48 | 7 |

A very large decrease in the capacity of the keratinocytes to bind hyaluronic acid is observed in the course of ageing, reflecting a loss of the CD44 functionality.

### EXAMPLE 3

Effect of the Combination of Gluconate and Calcium on the Binding of Hyaluronic Acid to Keratinocytes

The studies which follow were performed according to protocol I- of Example 1.

a) In a first experiment, the binding of hyaluronic acid labelled with a fluorochrome to human keratinocytes in culture in the presence of CaC₂ and calcium gluconate at equal molarity was compared.

The results are given in Table II below.

TABLE II

| Fluorescence measured (FU) | |
|---|---|
| CaCl₂ | Calcium gluconate |

| Concentrations | | Standard | | Standard |
|---|---|---|---|---|
| in mM | Mean | deviation | Mean | deviation |
| 5 | 30 | 9 | 310* | 30 |
| 10 | 370 | 21 | 2,745* | 125 |

*Highly significant differences between CaCl₂ and calcium gluconate.

It is observed that in the presence of calcium gluconate, the amount of fluorescence measured is significantly greater than for calcium chloride at an equivalent molarity. This indicates that much larger amounts of hyaluronic acid are bound to the keratinocyte cultures in the presence of this product than in the presence of calcium chloride.

b) In a second experiment, the action of increasing concentrations of CaCl₂ in the presence or absence of 10 mM sodium gluconate was compared.

The results are given in Table III below.

TABLE III

| | Fluorescence measured (FU) | |
|---|---|---|
| | Without sodium | With sodium |
| CaCl₂ | gluconate | gluconate (10 mM) |

| concentrations | | Standard | | Standard |
|---|---|---|---|---|
| in mM | Mean | deviation | Mean | deviation |
| 0 | 6 | 11 | 6 | 11 |
| 2.5 | 8 | 15 | 27 | 36 |
| 5 | 7 | 16 | 84* | 41 |
| 10 | 447 | 43 | 2,186* | 350 |

*Highly significant differences between CaCl₂ alone and in the presence of sodium gluconate.

This result confirms the positive effect of calcium on the binding of hyaluronic acid. The positive effect of calcium is manifested at a concentration of 10 mM.

In the presence of gluconate in the form of sodium gluconate, the amount of hyaluronic acid bound with 10 mM of CaCl₂ is greater. Moreover, at a CaCl₂ concentration of 5 mM, with no effect on this binding, the addition of sodium gluconate makes it possible to observe a significant binding of hyaluronic acid.

c) In a third experiment, the calcium concentration was set at 10 mM and the concentration of gluconate supplied in the form of the sodium salt was varied.

The results are given in Table IV below.

TABLE IV

| | |
|---|---|
| Sodium gluconate | Fluorescence measured (FU) |
| concentration | in the presence of 10 mM of CaCl₂ |

| in mM | Mean | Standard deviation |
|---|---|---|
| 0 | 447 | 43 |
| 2.5 | 5,241* | 597 |
| 5 | 4,535* | 616 |
| 10 | 2,186* | 350 |

*Highly significant differences between CaCl₂ alone and in the presence of sodium gluconate.

It is thus clearly seen that gluconate supplied in the form of sodium gluconate induces a dose-dependent potentiation of the effect of calcium and thus allows the binding of a much larger amount of hyaluronic acid than in the presence of calcium alone. This experiment shows that preferential Ca/gluconate molar ratios of close to 4 exist (10 mM/2.5 mM).

This last experiment is useful for adjusting the amount of gluconate to that of calcium to produce the maximum action.

It is seen that under relatively optimized conditions, sodium gluconate makes it possible at least to increase the effects of calcium by a factor of 12, which is considerable.

These last two elements are important since sodium gluconate may potentiate the effect of calcium, which is present in abundance in the epidermis, on the binding of hyaluronic acid to the surface of the keratinocytes in the epidermis.

It is thus seen that the invention is not limited only to calcium gluconate, but also relates more generally to gluconate in the form of a salt or a complex with calcium, magnesium, manganese, zinc or sodium or with any other monovalent or divalent cation.

### EXAMPLE 4

Effect of Sodium Lactate on the Expression of CD44 at the Surface of Human Keratinocytes in Culture, and on the Binding of Hyaluronic Acid by Human Keratinocytes in Culture

a) Expression of CD44 at the surface of human keratinocytes in the presence or absence of sodium lactate

This study was performed using the tests whose protocols are given in Example 1.

The study was performed on cultures of normal human keratinocytes.

The results are given in Table V below.

TABLE V

| | |
|---|---|
| | Expression of CD44 at the surface of |
| Sodium lactate | human keratinocytes |
| Concentrations | CD44 (FU)/cellular DNA (FU) labelling |

| in mM | Mean | % increase |
|---|---|---|
| 0 | 189 ± 28 | |
| 0.22 | 249 ± 31 | 32% |

In this experiment, it is observed that the fluorescence values corresponding to the labelling of the CD44 of the keratinocytes are increased when the cells are treated for 48 hours with a sodium lactate concentration of 0.22 mM.

b) Binding of hyaluronic acid to the surface of human keratinocytes in the presence or absence of sodium lactate.

This study was performed using the tests whose protocols are given in Example 1.

The study was performed on cultures of normal human keratinocytes.

The results are given in Table VI below.

TABLE VI

| | |
|---|---|
| | Binding of hyaluronic acid to the surface |
| Sodium lactate | of human keratinocytes |
| Concentrations | Binding (FU)/cellular DNA (FU) |

| in mM | Mean | % increase |
|---|---|---|
| 0 | 59 ± 8.5 | |
| 0.22 | 105 ± 24 | 78% |

In this experiment, it is demonstrated that the increase in the expression of CD44 of the keratinocytes at a sodium lactate concentration of 0.22 mM 25 (see Table VI above) is accompanied by an increase in the capacity of the keratinocytes to bind hyaluronic acid.

It will be noted that the activation of the binding of hyaluronic acid is greater than the increase in the expression of the receptor, which would suggest that sodium lactate may also have a favourable action on the functionality of the receptor.

Comment: In order to take account of a possible effect of the test substance on the number of cells and consequently on the number of receptors, the fluorescence values corresponding to the labelling of CD44 or to the binding of hyaluronic acid were normalized relative to the amount of cell material represented by the DNA values present in the cultures.

### EXAMPLE 5

Effect of Manganese Chloride on the Binding of Hyaluronic Acid by Human Keratinocytes in Culture

In this experiment, the binding of fluorescent hyaluronic acid to human keratinocytes in culture in the presence of CaCl₂ or in the presence of MnCl₂ at equivalent molarity was compared by applying the protocol given in Example 1.

| Fluorescence measured (FU) | |
|---|---|
| CaCl₂ | MnCl₂ |

| Concentrations | | Standard | | Standard |
|---|---|---|---|---|
| in mM | Mean | deviation | Mean | deviation |
| 5 | 37 | 26 | 1,593* | 421 |
| 10 | 361 | 144 | 1,524* | 397 |

*Significant differences in binding between CaCl₂ and calcium gluconate.

It is observed that in the presence of MnCl₂, the amount of fluorescence measured is significantly greater than for calcium chloride at equivalent molarity. This indicates that much larger amounts of hyaluronic acid are bound to the keratinocyte cultures in the presence of this product than in the presence of calcium chloride.

Examples of Cosmetic Formulations Using the Properties of the Agents for Stimulating the Binding of Hyaluronic Acid to Skin Cells

### EXAMPLE 6

### Moisturizing and Nourishing Anti-Age Emulsion

Calcium Gluconate . . . 0.1 g

Vitamin A palmitate . . . 0.001 g

Vitamin E phosphate . . . 0.01 g

Vitamin C magnesium phosphate . . . 0.2 g

Wheat ceramides . . . 0.2 g

Wheat proteins . . . 1 g

UVA-UVB screening agent . . . 5 g

Excipient with preserving agents and fragrances . . . qs100 g

This nourishing emulsion improves the hydration of the skin, in particular of the epidermis, the fineness and grain of the skin, and fades away wrinkles. It is applied daily to the face.

### EXAMPLE 7

### Firming and Restructuring Moisturizing Gel

Calcium gluconate . . . 0.2 g

Magnesium aspartate . . . 0.1 g

Titrated extract of Centella asiatica . . . 0.1 g

Soybean saponins . . . 0.05 g

Vitamin C magnesium phosphate . . . 0.1 g

Excipient with preserving agents and fragrances . . . qs100 g

This moisturizing gel exerts a tensioning effect on the skin and improves its suppleness and firmness. It is applied daily to the face, the neck and the neckline.

### EXAMPLE 8

### Moisturizing, Repairing Liposomal Gel

Sodium lactate . . . 0.15 g

Soybean lecithin . . . 2 g

Vitamin A acetate . . . 0.01 g

β-Ecdysone . . . 0.1 g

α-Tocopherol . . . 0.01 g

Excipient with preserving agents and fragrances . . . qs100 g

This liposomal gel is used in daily application, preferably in the evening, and on the face. It improves the suppleness of the skin and its moisturization, and promotes its regeneration.

### EXAMPLE 9

### Moisturizing and Tonifying Lotion,

Calcium gluconate . . . 0.1 g

Extract of Panax Ginseng . . . 0.2 g

Cyclic AMP . . . 05 g

Caffeine . . . 0.1 g

Excipient with preserving agents and fragrances . . . qs100 g

This moisturizing lotion is used in daily application on the face, preferably in the morning, to obtain a more beautiful and more radiant skin.

### EXAMPLE 10

### Moisturizing and Calmant Fluid

Sodium gluconate . . . 0.25 g

Magnesium chloride . . . 0.1 g

D-Xylose . . . 0.2 g

Wheat ceramides . . . 0.2 g

Extract of liquorice . . . 0.1 g

Excipient with preserving agents and fragrances . . . qs100 g

This fluid is used as a daily topical application on the face and the body.

### EXAMPLE 11

### Moisturizing Mascara

Calcium gluconate . . . 0.3 g

Hyaluronic acid . . . 0.5 g

D-Xylose . . . 0.3 g

Coloured pigments . . . 10 g

Waxes . . . 30 g

Excipient . . . qs 100 g

### EXAMPLE 12

### Patch for Soothing After Sunburn

Magnesium gluconate . . . 0.5 g

Ammonium glycyrrhizinate . . . 0.5 g

Dextran sulphate . . . 0.2 g

Excipient for a patch . . . qs 100 g

### EXAMPLE 13

### Antiseptic and Soothing Cream for Chapping, Small Surface Wounds and Irritation Redness

Manganese chloride . . . 0.1 g

Extract of Phellodendron amurense . . . 0.1 g

β-Ecdysone . . . 0.1 g

Fusidic acid . . . 2 g

Excipient . . . qs 100 g

1. Use, in a cosmetic composition, of at least one active agent which increases the expression of the CD44 membrane receptors of skin cells and/or which improves the binding to the surface of the said skin cells of hyaluronic acid, and/or of collagen, in particular of collagen I and/or collagen IV, and/or fibronectin.

2. Use according to claim 1, characterized in that the said active agent increases the expression of the CD44 receptors of keratinocytes and/or of fibroblasts and/or improves the binding of hyaluronic acid to the surface of the said cells.

3. Use according to claim 1, characterized in that the said active agent increases the expression of the CD44 receptors of fibroblasts and/or improves the binding to the surface of the said fibroblasts, of collagen, in particular of collagen I and/or collagen IV, and/or of fibronectin.

4. Use according to one of claims 1 to 3, characterized in that the said active agent is chosen from the group consisting of cosmetically acceptable α-hydroxy acids, which are preferably C₂-C₁₂, salts thereof and esters thereof with a C₂-C₂₄ and preferably C₁₄-C₂₂ alcohol.

5. Use according to claim 4, characterized in that the said α-hydroxy acid is chosen from the group consisting of lactic acid, glycolic acid, gentisic acid, salicylic acid, gluconic acid and heptonic acid.

6. Use according the claim 4, characterized in that the said cosmetically acceptable α-hydroxy acid salt is chosen from the group consisting of sodium, calcium, magnesium, zinc and manganese salts.

7. Use according to one of claims 1 to 6, characterized in that the said active agent is calcium gluconate or a gluconic acid salt combined with a calcium salt, for example with calcium chloride.

8. Use according to claim 7, characterized in that the molar ratio of calcium to gluconate is between 2 and 6 and is preferably about 4.

9. Use according to one of claims 1 to 3, characterized in that the said active agent is chosen from the group consisting of cosmetically acceptable α-keto acids, which are preferably C₃-C₁₂, salts thereof and esters thereof with a C₂-C₂₄ and preferably C₁₄-C₂₂ alcohol.

10. Use according to claim 9, characterized in that the said α-keto acid is pyruvic acid.

11. Use according to claim 9 or 10, characterized in that the said α-keto acid salt is chosen from the group consisting of the sodium, calcium, magnesium, zinc and manganese salts.

12. Use according to one of claims 1 to 3, characterized in that the said active agent is manganese chloride.

13. Use according to one of claims 1 to 11, characterized in that the proportion of the said active agent in the composition is between 0.005% and 5% by weight and preferably between 0.05% and 0.5% by weight relative to the total weight of the final cosmetic composition.

14. Use according to one of claims 1 to 13, characterized in that the said active agent is used in the composition in combination with at least one substance chosen from the group consisting of vitamins, in particular vitamins of group. A (retinol), C and D3 and derivatives thereof such as esters, in particular the palmitates and propionates, tocopherols, xanthines, in particular caffeine or theophylline, retinoids, in particular vitamin A acid, extracts of Centella asiatica, asiatic acid and madecassic acid and glycosyl derivatives thereof, such as asiaticoside or madecassoside, extracts of Siegesbeckia orientalis, extracts of Commiphora mukul and extracts of Eriobotrya japonica, cosmetically acceptable silicon derivatives such as polysiloxanes, silanols and silicones, amino acids and salts thereof, especially the magnesium or calcium salts, in particular aspartic acid, arginine, citrulline and threonine, ceramides, glycoceramides, sphingosine derivatives, in particular ceramides of type II and type III, phospholipids, forskolin and derivatives thereof, extracts of Coleus, extracts of Tephrosia, elastase inhibitors, in particular ellagic acid, soybean peptides, collagenase inhibitors, in particular peptides and plant extracts such as extracts of Coptidis root, extracts of Scutellaria baicalensis Georgi root, flavonoids such as wogonine, baicaline and baicaleine, aqueous-ethanolic extracts of leaves of Ginkgo biloba, of Mosla chinensis, of Salvia officinalis, of Cinnamomum cassia, cathechic extracts of Camellia sinensis and aqueous extracts of Theobroma cacao bean husks, extracts of Phellodendron amurense, anti-inflammatory agents, in particular phospholipase A2 inhibitors, calmants, in particular extracts of liquorice, glycyrrhetinic acid, ammonium glycyrrhizinate, moisturizers, in particular polyols, propylene glycol, butylene glycol, glycerol, hyaluronic acid, anti-stretch mark agents, in particular extracts of common horse chestnut and escin, agents for protecting or improving the microcirculation, in particular bioflavonoids from Ginkgo biloba, isodon, extracts of Ami visnaga, visnadine, ruscogenin, free-radical scavengers, in particular polyphenols such as PCOs (Procyanidol Oligomers) and derivatives thereof, plant extracts, in particular extracts of Curcuma longa, anti-seborrhoeic agents such as a 5-α-reductase inhibitor, in particular an extract of Pygeum africanum, and agents for stimulating the blood microcirculation, such as cepharanthine and methyl nicotinate, alfalfa saponins, soya sapogenols, in particular of type A and type B, agents for stimulating collagen VII synthesis, such as extracts of Bertholletia, ellagic acid, D-xylose and trace element complexes.

15. Use according to one of claims 1 to 14, characterized in that the said active agent is used in the composition in combination with at least one substance chosen from substances for protecting the skin against the harmful effects of sunlight, such as sunscreens alone or in combination, in particular UVA screening agents and UVB screening agents, in particular titanium oxides and zinc oxides, oxybenzone, cinnamates, in particular octyl methoxycinnamate, butylmethoxydibenzoylmethane and screening agents of plant origin, substances for limiting the damage caused by DNA, in particular those limiting the formation of thymine dimers, such as ascorbic acid and derivatives thereof and/or Photonyl, and substances for contributing towards removing age marks, such as inhibitors of melanin synthesis or tyrosinase inhibitors.

16. Cosmetic care method, characterized in that it comprises the application to the areas of skin to be treated of a cosmetic composition containing, as active principle, at least one agent for increasing the expression of the CD44 membrane receptors of skin cells and/or for improving the binding to the surface of the said skin cells of hyaluronic acid, and/or of collagen, in particular of collagen I and/or collagen IV, and/or of fibronectin.

17. Cosmetic care method according to claim 16, characterized in that the said agent is an active agent as defined in one of claims 4 to 12, chosen from cosmetically acceptable α-hydroxy acids, salts thereof and esters thereof with a C₂-C₂₄ and preferably C₁₄-C₂₂ alcohol, cosmetically acceptable α-keto acids, salts thereof and esters thereof with a C₂-C₂₄ and preferably C₁₄-C₂₂ alcohol, and manganese chloride.

18. Cosmetic care method according to claim 17, characterized in that it is intended to correct the deficiency in the binding, to the surface of the said cells, of hyaluronic acid and/or of collagen, in particular of collagen I and/or collagen IV, and/or of fibronectin.

19. Cosmetic care method according to one of claims 17 and 18, characterized in that it is intended to improve the moisturization of the epidermis.

20. Method according to one of claims 17 to 19, characterized in that it is intended treating sensitive or reactive skin.

21. Method according to one of claims 17 to 19, characterized in that it is intended to correct the negative effects of ageing on the expression of the CD44 membrane receptors of skin cells and/or on the binding to the surface of the said skin cells of hyaluronic acid and/or of collagen, in particular of collagen I and/or collagen IV, and/or of fibronectin.

22. Use of at least one active agent chosen from pharmaceutically acceptable α-hydroxy acids, salts thereof and esters thereof with a C₂-C₂₄ and preferably Cl₄-C₂₂ alcohol, pharmaceutically acceptable α-keto acids, salts thereof and esters thereof, with a C₂-C₂₄ and preferably C₁₄-C₂₂ alcohol, and manganese chloride, for the preparation of a pharmaceutical composition for topical use which is intended for treating a deficiency in the expression of the CD44 receptors of the skin and/or their ability to bind hyaluronic acid and/or collagen, in particular collagen I and/or collagen IV, and/or fibronectin to the surface of the said cells.

23. Use according to claim 22, characterized in that the said composition is intended for preventing and/or treating inflammatory phenomena.
